(19) United States
(12) Patent Application Publication
(10) Pub. No.: US 2026/0297629 A1
(43) Pub. Date: Oct. 1, 2026

(54) PHOTOBIOLOGICAL HYDROGEN PRODUCTION METHOD BASED ON CELL ENGINEERING

(71) Applicant: INDUSTRY-ACADEMIC COOPERATION FOUNDATION, YONSEI UNIVERSITY, Seoul (KR)

(72) Inventors: Hyun Seo AHN, Seoul (KR); Won Hyoung RYU, Goyang-si, Gyeonggi-do (KR); Hyo Jin GWON, Seoul (KR); Jae Hyoung YUN, Seoul (KR)

(21) Appl. No.: 19/479,465

(22) PCT Filed: Apr. 29, 2024
(86) PCT No.: PCT/KR2024/005765

§ 371 Date: Oct. 28, 2025

(30) Foreign Application Priority Data
Apr. 28, 2023 (KR) 10-2023-0056016

(51) Int. Cl. C12P 3/00; C12N 1/12; C12N 5/04; C12N 13/00; C12R 1/89

(52) U.S. Cl. CPC C12P 3/00; C12N 1/12; C12N 5/04; C12N 13/00; C12N 2510/02; C12N 2529/10; C12R 2001/89

(57) ABSTRACT

According to one embodiment, an engineered cell comprises a chloroplast-containing cell and a conductor inserted into the cell, wherein the conductor is inserted so as to contact or penetrate the thylakoid membrane of the chloroplast, whereby the engineered cell is engineered so that photosynthetic electrons can be extracted out of the cell by light; a metal catalyst using the engineered cell; and a hydrogen production method using the engineered cell. By using the metal catalyst, oxygen generated during photosynthesis can be catalyzed and self-regulated at a low concentration and protons can be catalyzed to provide a hydrogen production system and method that enable sustained hydrogen production.

### TECHNICAL FIELD

The present invention relates to a cell comprising a chloroplast; and a wire inserted into the cell, wherein the wire is inserted so as to contact or penetrate the thylakoid membrane of the chloroplast, and is manipulated such that photosynthetic electrons can be extracted from the cell by light, and to a method for producing a metal catalyst and hydrogen using the same.

### BACKGROUND ART

Due to the depletion of fossil fuels and environmental pollution problems, the demand for sustainable energy sources has been increasing, and interest in solar energy has also been rapidly growing. However, since solar energy is difficult to use directly, continuous research has been conducted to convert it into other forms of energy such as geothermal or electric power. Among various energy sources, hydrogen fuel has the advantage of being carbon-free and reusable. It also has the highest energy density, and its product is pure water, making it environmentally friendly. One of the methods for storing solar energy as hydrogen involves a renewable and biologically tunable biological solar-to-hydrogen conversion process, which has been actively studied.

Recently, biological solar-to-hydrogen conversion methods have attracted attention, particularly hydrogen production using hydrogenase-expressing organisms represented by green algae, cyanobacteria, and purple photosynthetic bacteria. Representative examples include solar-to-hydrogen conversion using [FeFe]-hydrogenase in green algae and [NiFe]-hydrogenase in cyanobacteria. However, hydrogen production using photosystem II within the organism accompanies the generation of oxygen (O₂), and the produced oxygen deteriorates the function of hydrogenase. Therefore, it is necessary to immediately create an anaerobic environment while irradiating light to the organism, or to perform molecular biological manipulation to develop oxygen-resistant (oxygen-insensitive) hydrogenases. Accordingly, in order to improve the efficiency of converting solar energy to hydrogen energy while using water as an electron donor, there is a need for a novel hydrogen production method different from the existing approaches.

As a result of efforts to develop an efficient new photobiological hydrogen production method capable of converting solar energy into fuel, the inventors of the present invention have completed a manipulated cell in which a wire is inserted into a cell containing a chloroplast so that photosynthetic electrons of the chloroplast can be extracted outside the cell, and a hydrogen production system using the same.

### DETAILED DESCRIPTION OF THE INVENTION

### Technical Problem

The object of the present invention is to provide a manipulated cell comprising a cell including a chloroplast and a wire inserted into the cell, wherein the wire is inserted so as to contact or penetrate the thylakoid membrane of the chloroplast so that photosynthetic electrons can be extracted from the cell by light energy, and to provide a method for manufacturing the same.

Another object of the present invention is to provide a metal catalyst comprising metal particles formed on the surface of a wire by irradiating light to a cell comprising a chloroplast and a wire manipulated such that the wire is inserted from outside the cell to the inside of the chloroplast.

Still another object of the present invention is to provide a method for producing hydrogen and a hydrogen production system using a manipulated cell comprising a cell including a chloroplast and a wire inserted therein, wherein the wire is inserted so as to contact or penetrate the thylakoid membrane of the chloroplast such that photosynthetic electrons can be extracted from the cell by light energy.

### Technical Solution

In order to achieve the above objects, one aspect of the present invention provides a manipulated cell comprising a cell including a chloroplast and a wire inserted into the cell, wherein the wire is manipulated such that it is inserted from outside the cell to the inside of the chloroplast.

the present invention also provides a method for manufacturing a manipulated cell, comprising: a) preparing a dispersion containing a cell culture including chloroplasts and a wire; b) adding the dispersion containing the wire to the cell culture to prepare a mixed solution; and c) inserting the wire into the inside of the cell through physical collision between the cells and the wire in the mixed solution.

Another aspect of the present invention provides a metal catalyst comprising a wire and metal particles formed on the surface of the wire, wherein the metal particles are formed by photosynthetic electrons generated from a cell when light is irradiated to a mixture of a metal ion solution and a cell including a chloroplast and manipulated such that the wire is inserted from outside the cell to the inside of the chloroplast.

The present invention further provides a method for producing a metal catalyst, comprising: a) irradiating light to a mixture of a cell including a chloroplast and manipulated such that a wire is inserted from outside the cell to the inside of the chloroplast, and a metal ion solution, thereby forming metal particles on the surface of the wire; and b) separating the wire on which the metal particles are formed.

Another aspect of the present invention provides a method for producing hydrogen, comprising irradiating light to a mixture of a cell including a chloroplast and manipulated such that the wire is inserted from outside the cell to the inside of the chloroplast, and a metal ion solution, thereby producing hydrogen.

In addition, the present invention provides a hydrogen production system comprising: a photosynthesis induction unit including a cell comprising a chloroplast and manipulated such that a wire is inserted from outside the cell to the inside of the chloroplast, a normal cell including a chloroplast, and a metal ion solution; a light supply unit; a CO₂ supply unit; and a sealed hydrogen storage unit.

In the present invention, the wire may contact or penetrate the thylakoid membrane of the chloroplast.

In addition, the wire may be made of a carbon nanomaterial or a conductive metal material, and the conductive metal may be selected from the group consisting of gold, platinum, copper, silver, iron, nickel, cobalt, and lead.

Furthermore, the diameter of the wire may be 100 to 400 nm, and the length of the wire may be 5 to 30 m.

In the present invention, the cell may be selected from the group consisting of algal cells, plant cells, and artificial cells containing chloroplasts.

In the present invention, the manipulated cell may be characterized in that photosynthetic electrons generated inside the cell by light are transferred to the outside of the cell through the wire.

At this time, the light may be irradiated at a light intensity of 5 to 15 mW/cm² and for 5 to 20 hours.

In the method for manufacturing the manipulated cell according to the present invention, insertion of the wire into the inside of the cell containing the chloroplast may be performed by fluid injection or ball milling, and any other possible method for inserting the wire into the inside of the cell through physical collision may be used.

In the hydrogen production method according to the present invention, the method may comprise: a) a step of extracting photosynthetic electrons generated inside the cell by light through the wire to the outside of the cell; b) a step of forming metal ions on the surface of the wire by the extracted photosynthetic electrons; and c) a step of generating hydrogen on the surface of the wire where the metal ions are formed.

At this time, in the initial stage of step c), oxygen in the metal ion solution may be removed by an oxygen reduction reaction, and the hydrogen generation reaction may occur after the oxygen is removed.

In the hydrogen production system according to the present invention, the normal cell containing chloroplasts and the manipulated cell may be present at a ratio of 1:1 to 3, and the normal cell containing chloroplasts and the manipulated cell may be suspended in the metal ion solution.

In addition, after step b), the method may further comprise a step of removing the metal ion solution and replacing it with a clean culture solution for hydrogen production that does not contain metal ions. This is because sufficient metal ions have been formed on the surface of the wire through step b), and the step is intended to prevent unnecessary continuous metal ion reduction reactions and to improve the efficiency of the hydrogen production reaction.

### Advantageous Effects

According to the present invention, a manipulated cell is provided in which a wire is inserted into a cell containing a chloroplast so that photosynthetic electrons of the cell can be extracted outside the cell, and a metal catalyst utilizing the photosynthetic electrons of such a manipulated cell can be provided. Furthermore, a hydrogen production system and method capable of continuously producing hydrogen by catalyzing oxygen generated during photosynthesis to self-control at a low concentration and catalyzing protons can be provided by using the same.

In addition, according to the present invention, solar energy can be converted into fuel by consuming only CO₂ and water as the electron sources, and in particular, an artificial photosynthesis system excellent in cost efficiency and immediate availability of the produced fuel can be provided.

### DESCRIPTION OF THE DRAWINGS

FIG. 1 schematically shows a cell system for hydrogen production according to an embodiment of the present invention and a method for producing hydrogen using the same.

FIG. 2 is an optical image showing a form of carbon nanofiber insertion into green algal cells used in the present invention.

FIG. 3 is an optical image showing a form of carbon nanofiber insertion into diatom cells used in the present invention.

FIG. 4 is an optical image showing the form of carbon nanofiber insertion according to the type of wire used in the present invention.

FIG. 5 shows a dispersion of carbon nanorods.

FIG. 6 schematically shows a method for introducing carbon nanorods into plant cells.

FIG. 7 shows the insertion rate into cells according to the adjustment of the diameter of carbon nanofibers.

FIG. 8 is an analysis of photosynthetic current extraction on a single-cell basis.

FIG. 9 shows the result of confirming whether metal ion particles were formed on the carbon nanorods exposed outside the plant cell after irradiating light to a mixture of the plant cell into which carbon nanorods were introduced and a metal ion solution.

FIG. 10 shows the formation of metal ion particles on carbon nanorods and the cell death caused by the cytotoxicity of copper and silver.

FIG. 11 is a schematic diagram of the energy transfer and storage pathway in the artificial photosynthesis system according to the present invention.

FIG. 12 shows a configuration in which the plant cell is arranged in a sealed batch after metal ion particles are formed on the carbon nanorods exposed outside the plant cell.

FIG. 13 shows the results of measuring the oxygen concentration inside the sealed batch and the hydrogen concentration produced over time during light irradiation.

FIG. 14 shows the change in hydrogen production according to the optimization of the ratio of engineered cells and the batch volume.

FIG. 15 shows the effect of CO₂ reduction through photosynthetic CO₂ fixation.

### BEST MODE

To achieve the above object, one aspect of the present invention provides a manipulated cell comprising a cell including a chloroplast and a wire inserted into the cell, wherein the wire is inserted so as to contact or penetrate the thylakoid membrane of the chloroplast so that photosynthetic electrons can be extracted from the cell by light energy.

A chloroplast is an organelle for photosynthesis in eukaryotic cells and has a double membrane composed of an outer membrane and an inner membrane, and the interior thereof contains flattened thylakoids. The exterior of the thylakoid is filled with a stroma, which is the matrix. The light reaction occurs on the thylakoid membrane, and water is decomposed to release oxygen while generating NADPH and ATP. The dark reaction reduces carbon dioxide to synthesize glucose (C₆H₁₂O₆) using NADPH and ATP, which are products of the light reaction. In the present invention, electrons generated during the photosynthetic process of the chloroplast can be extracted outside the cell through a wire that connects the thylakoid membrane of the chloroplast to the outside of the cell.

In the present invention, the term “wire (electric wire, chord)” refers to a conductor formed into a long and thin line to allow electric current to flow, and the term “electric wire” may also be used. The wire may have the form of a conductive tube, and the term “conductive tube” refers to a tube-shaped structure made of a conductive material.

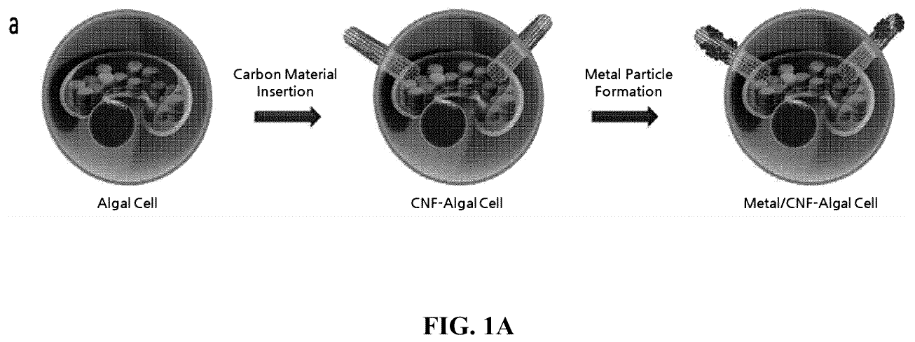

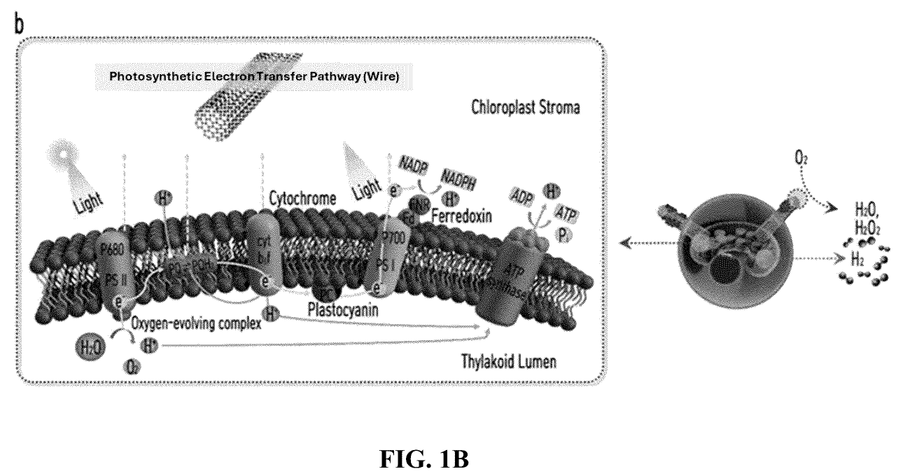

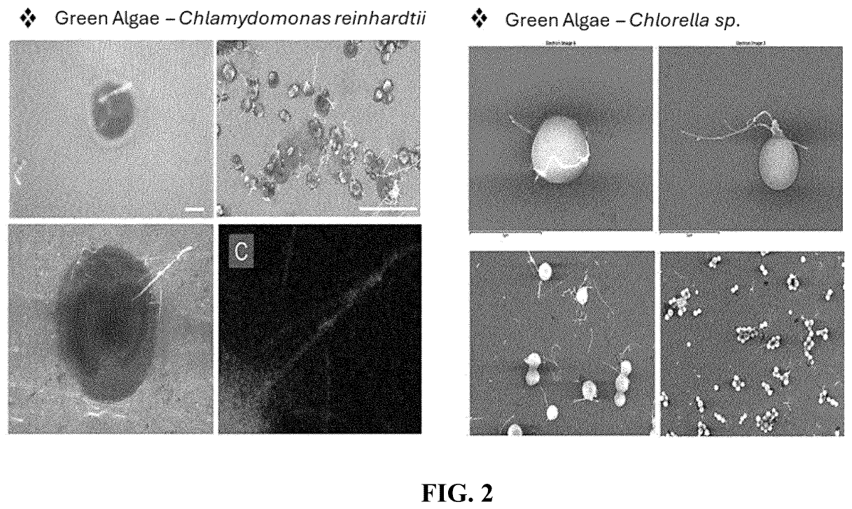

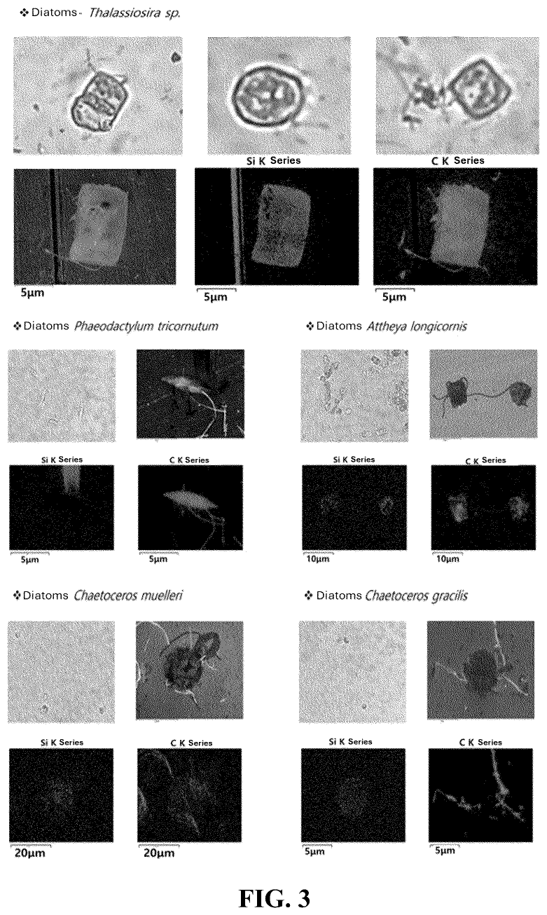

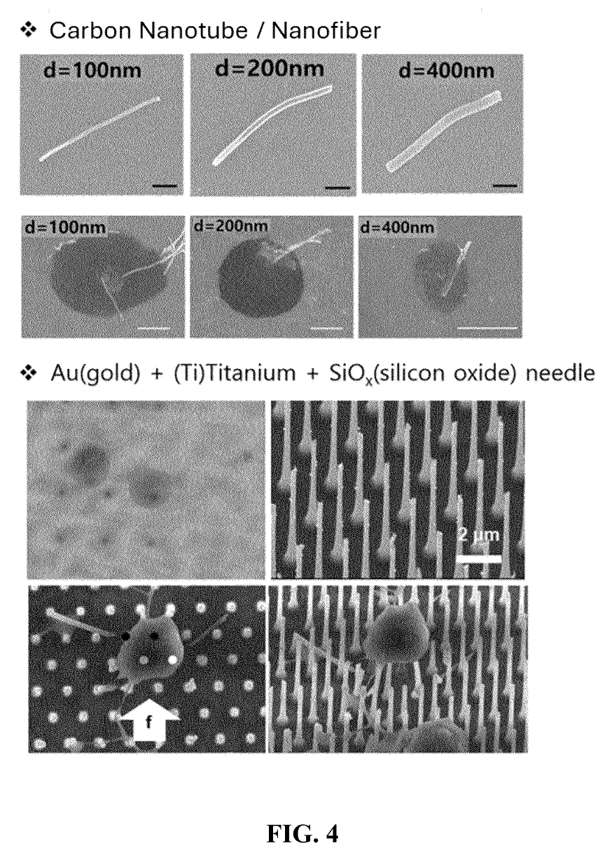

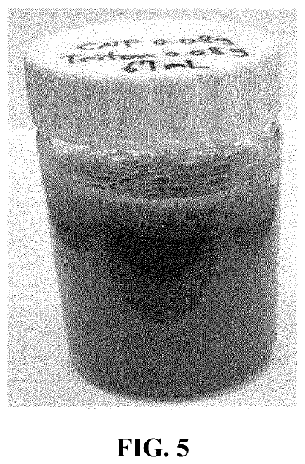

As shown in FIG. 1, the wire or conductive tube is inserted from outside the cell to the inside of the chloroplast, connecting the inside of the chloroplast with the outside of the cell, and serves to extract photosynthetic electrons outside the cell. At this time, the wire or conductive tube may contact or penetrate the thylakoid membrane of the chloroplast, and one or more wires or conductive tubes may contact a single thylakoid membrane. In addition, a portion of the wire or conductive tube is exposed outside the cell so that oxygen outside the cell can gain electrons and be reduced to water.

According to one embodiment of the present invention, the wire or conductive tube is made of carbon or a conductive metal, and the conductive metal may include platinum, gold, silver, copper, iron, nickel, cobalt, or lead, and various conductive metals can be used except for those that exhibit high cytotoxicity. In the present invention, a carbon nanofiber as a carbon-based conductive material and gold as a metal-type conductive material were used for insertion into the chloroplast, and particularly, the diameter and length of the carbon nanofiber were varied for insertion into the chloroplast to determine the optimal size.

According to one embodiment of the present invention, the conductive tube may have a diameter of 60 to 200 nm, preferably 60 to 150 nm, and more preferably 80 to 120 nm. When the diameter of the conductive tube is 50 nm or less, it is too small to be inserted into the cell, and when the diameter of the conductive tube is 300 nm or more, it may induce cell death.

According to one embodiment of the present invention, the wire or conductive tube may have a length of 5 to 30 m, preferably 5 to 20 m, and more preferably 8 to 15 m. If the conductive tube is too short, it cannot connect the chloroplast with the outside of the cell, and conversely, if it is too long, the insertion efficiency decreases.

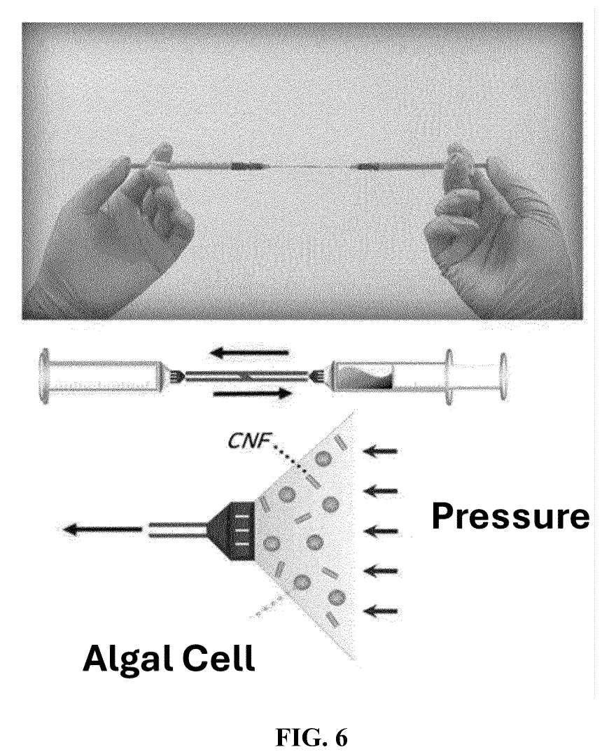

As shown in FIG. 6, the wire or conductive tube may be inserted into the cell by a method such as fluid injection or ball milling, and the number of wires introduced into the cell may be random but mostly one per cell. In the case of the fluid injection method, cells and wires existing separately in a large-diameter tube pass through a smaller-diameter tube, increasing effective physical collisions, and during this process, the wire is inserted into the cell. In the embodiment of the present invention, when using CNF (carbon nanofiber) with a length of 7 m and a diameter of 100 nm, it was confirmed that the conductive material was inserted into the cell with an efficiency of up to 94%, and this ratio could be improved by adjusting the ratio of cells to conductive materials.

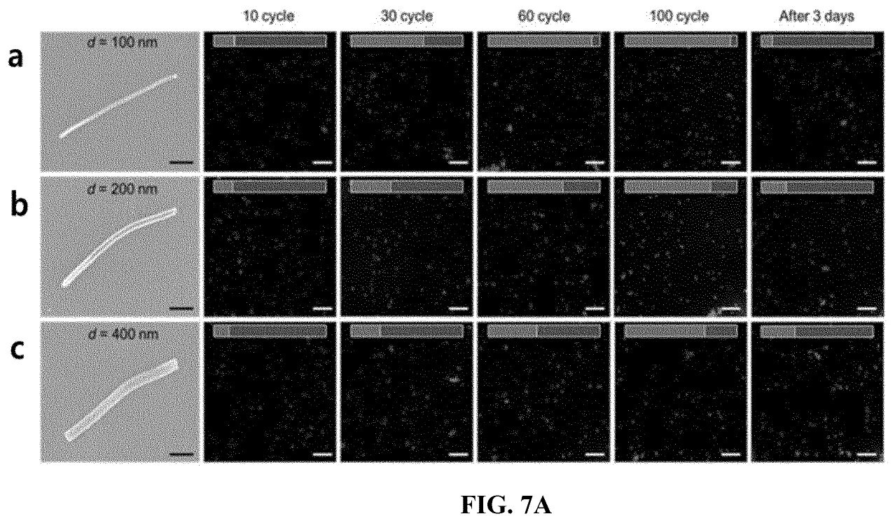

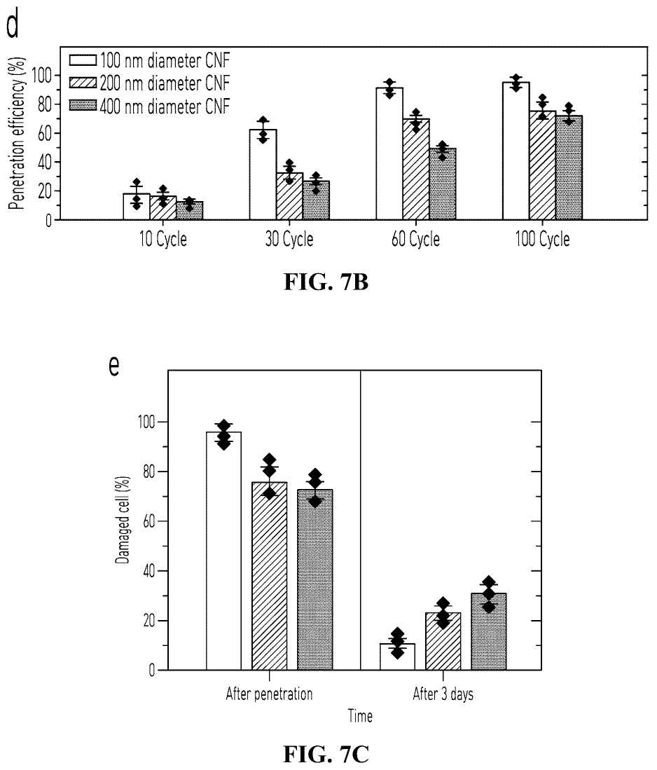

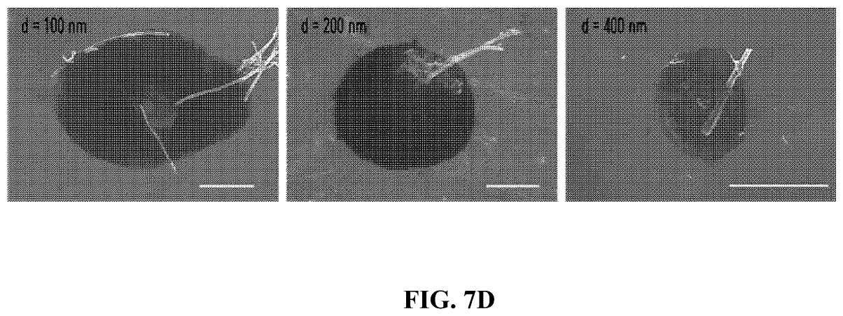

Furthermore, by varying the diameter of the carbon nanofiber and the number of injections, the degree of cell rupture by insertion and the degree of cell recovery after insertion were examined, and the insertion rate according to the diameter of the carbon nanofiber and the number of injections was confirmed. As shown in FIG. 7, the insertion rate of the carbon nanofiber having a diameter of 100 nm was the highest, and the insertion rate increased as the number of injections increased. It was also confirmed that after a lapse of time, the cells recovered and the degree of cell damage was the lowest.

According to one embodiment of the present invention, the cell may be a plant cell, an alga, or a photosynthetic bacterium, but is not limited thereto, and an artificial cell containing a chloroplast may also be used.

The algae include brown algae, green algae, and red algae. Algae have chloroplasts and perform photosynthesis by absorbing light of complementary color to their own body color. Green algae, brown algae, and red algae absorb red light (640-700 nm), yellow light (570-590 nm), and blue light (400-500 nm), respectively, to perform photosynthesis. The photosynthetic bacteria include cyanobacteria and purple photosynthetic bacteria. Since the photosynthetic bacteria have cell walls, the present invention can also be applied to cells having cell walls if a wire having strength, size, and shape sufficient to penetrate the cell wall and cell membrane is used.

Cyanobacteria are bacteria that produce oxygen through photosynthesis. Although they do not have differentiated cellular organelles such as nucleus, chloroplast, and mitochondria, they have chlorophyll a and assimilation pigments such as phycocyanin and phycoerythrin, which make photosynthesis possible. These assimilation pigments are located inside the thylakoid or on the surface of the thylakoid membrane.

Meanwhile, the cell may include not only a natural cell but also a modified cell. For example, a cell whose cell wall has been weakened by transformation, or an artificial cell into which a chloroplast or other photosynthetic organelle has been introduced, may also be used.

In the present invention, insertion of carbon nanofibers was confirmed using green algae of Chlorella sp., Chlamydomonas reinhardtii, and diatoms such as Chaetoceros sp., Phaeodactylum sp., Attheya sp., and Thalassiosira sp. Among these, Chlamydomonas reinhardtii was particularly suitable for use in the present invention because it has a long lifespan, a fast growth rate, and chloroplasts occupying more than 70% of the cell.

Another aspect of the present invention provides a metal catalyst by producing a mixture in which the manipulated cells are freely suspended in a metal ion solution and irradiating light thereto to induce a reduction reaction of metal ions caused by photosynthetic electrons extracted through the wire. In addition, the present invention provides a method and a system for hydrogen production by inducing oxygen reduction and hydrogen generation through photosynthetic electrons extracted from the manipulated cell.

In the present invention, the metal ions may be selected from the group consisting of platinum, copper, and silver, but are not limited thereto. In the embodiment of the present invention, a low concentration of platinum ions was used. The range of the term “low concentration” may vary depending on the degree of damage caused to the cells by the metal ions and the type of metal ions, and for example, may be within the range of 0.1 to 10 mM.

At this time, the manipulated cells may be provided in a state dispersed in a buffer capable of maintaining cellular function, and in the embodiment of the present invention, TAP medium suitable for green algae was used.

The hydrogen production method according to the present invention may be characterized in that hydrogen is produced through a hydrogen generation reaction by irradiating light to a mixture of the manipulated cells and a metal ion solution so that photosynthetic electrons extracted through the wire from the manipulated cells produce hydrogen, and collecting the generated hydrogen.

More specifically, the hydrogen production process consists of a step of forming metal catalyst particles on the surface of the wire inserted into the manipulated cell, a step of inducing an oxygen reduction reaction of dissolved oxygen in the aqueous solution on the surface of the metal catalyst particles, and a step of inducing a hydrogen generation reaction after the oxygen reduction reaction decreases.

The process of forming the metal catalyst particles proceeds as follows: when the photosynthetic electron transport chain in the thylakoid is activated by light irradiation and the energy level of the electrons flowing through the photosynthetic electron transport chain becomes greater than or equal to the energy level required for the reduction reaction of the metal ions present in the external solution, the reduction reaction of the metal ions occurs on the surface of the wire or conductive tube due to the photosynthetic electrons extracted from the wire or conductive tube. As a result of this reduction reaction, metals are coated or metal particles are formed on the surface of the wire or conductive tube exposed outside the cell. These metal catalysts catalyze the oxygen generated during photosynthesis to maintain oxygen at a low concentration by self-control and catalyze protons to enable continuous hydrogen production.

The light irradiation time may be performed for 5 to 20 hours, preferably 8 to 15 hours, and more preferably 12 hours. When the time is shorter than the above range, the reaction time sufficient for synthesizing metal particles cannot be ensured, resulting in poor reproducibility and consistency of the synthesis reaction. When the time is longer than the above range, the metal particles are sufficiently synthesized, but the penetration of metal ions dissolved in the aqueous solution into the cell may increase, adversely affecting cell viability. However, the light irradiation time may vary depending on the concentration of the hydrogen-producing cells and the concentration of the metal ions used, and the light may be irradiated at a suitable wavelength according to the type of pigments contained in the cells or chloroplasts used. For example, when green algae are used, red light of a long wavelength may be irradiated.

In addition, the light may be irradiated at a light intensity of 5 to 15, preferably 8 to 12, and more preferably 10 mW/cm². At a light intensity exceeding the above range, harmful reactive oxygen species may be generated in the green algal cells due to excessive light intensity, or a photobleaching effect, in which light is not sufficiently absorbed, may occur. Conversely, at a light intensity lower than the above range, the penetration rate of metal ions into the cell may dominate over the reduction reaction of metal ions by extracted electrons, adversely affecting cell survival. However, the light intensity may vary depending on the concentration of the hydrogen-producing cells and the concentration of the metal ions used.

The metal catalyst including metal particles formed on the surface of the wire can be separated from the cells by a centrifuge, and any other method capable of separating the metal catalyst and the cells can be used.

The conductive metals synthesized in the present invention are composed of gold, platinum, copper, silver, iron, nickel, cobalt, and lead, and can be applied to future catalyst industries in various fields.

For example, a polymer electrolyte fuel cell is a device that converts the chemical energy of fuel into electrical energy by electrochemically reacting fuel such as hydrogen or methanol with oxygen, and since it has a low operating temperature and high power generation efficiency, it is considered promising for use in small-scale power generation devices such as portable electronic devices and as a power source for electric vehicles. In the polymer electrolyte fuel cell, fuel such as hydrogen or methanol is oxidized at the anode, and air or oxygen is reduced at the cathode to generate water. A catalyst with high activity, such as platinum nanoparticles supported on a carbon material, is used as the oxidation-reduction catalyst. The platinum metal catalyst combined with the carbon nanofiber according to the present invention can be actively utilized in the field of fuel cell catalysts.

According to the embodiment of the present invention, since the synthesis of metal catalyst particles occurs on the wire inserted into the cell containing chloroplasts, a composite catalyst containing metal particles on the surface of the wire can be easily manufactured. As a result, the process of manufacturing a metal catalyst and a composite catalyst deposited on the surface of the wire can be simplified, and production costs can be reduced, facilitating mass production. It can be applied to electrochemical and catalytic chemistry fields including electrodes of secondary batteries, fuel cells, and supercapacitors.

The oxygen reduction reaction and hydrogen generation reaction occur when photosynthetic electrons extracted by light irradiation from a manipulated cell in which metal catalyst particles are formed on a wire or a conductive tube induce an oxygen reduction reaction of the dissolved oxygen on the surface of the metal catalyst particles. As a result, the oxygen concentration inside the sealed batch decreases, and newly generated oxygen by photosynthesis is continuously removed. Consequently, the oxygen concentration inside the sealed batch can be maintained at a low fraction of about 1%.

The hydrogen production process may be performed by separating the manipulated cell, in which metal catalyst particles are formed on the wire or conductive tube, and transferring it to a sealed batch, and hydrogen generated in a separated space where no inflow or outflow of air occurs can be collected. Alternatively, the process may be continuously performed without transferring to a sealed batch if hydrogen generated in a structure isolated from external air can be obtained.

Here, the term “sealed batch” refers to a structure in which the inflow and outflow of air from outside cannot occur and which contains manipulated cells for hydrogen production dispersed in a cell culture solution or buffer. The remaining space contains ordinary air. In addition, the sealed batch may further include a configuration capable of replacing the cell culture medium (for example, TAP) or extracting the generated hydrogen gas.

The light may be irradiated at an intensity of 0.1 to 5, preferably 0.5 to 4, and more preferably 2 mW/cm². At this time, light is continuously irradiated to the sealed batch for hydrogen production. In order for cells receiving continuous light to maintain high photosynthetic efficiency over a long period of time while producing hydrogen, 1) the light intensity must be sufficient for the cells to fully absorb the light, and 2) the light intensity must not be excessive for the cellular photosynthetic system (typically 2-10 mW/cm²). The inventors determined the required light intensity for hydrogen production within the above range considering the photosynthetic activity of the cells.

According to the present invention, the extracted photosynthetic electrons induce an oxygen reduction reaction on the surface of the metal catalyst particles, thereby removing oxygen present in the aqueous solution and newly generated by photosynthesis and self-controlling the oxygen concentration to a low level, followed by a hydrogen evolution reaction. Through such self-control of oxygen, hydrogen fuel can be used without additional oxygen separation or purification.

### Cell Culture

Chlamydomonas reinhardtii cells were cultured in Tris/acetate/phosphate (TAP) liquid medium composed of the following components: 380 mg/LNH₄Cl, 10 mg/L (NH₄)₆Mo₇O₂₄·4H₂O, 10 mg/L H₃BO₃, 50 mg/L CaCl₂ 2H₂O, 20 mg/L CoCl₂·6H₂O, 20 mg/L CuSO₄·5H₂O, 50 mg/L FeSO₄·7H₂O, 100 mg/L MgSO₄·7H₂O, 50 mg/L MnCl₂ 4H₂O, 50 mg/L KH₂PO₄, 110 mg/L K₂HPO₄, 20 mg/L ZnSO₄ 7H₂O, 50 mg/L EDTA, 1100 mg/L glacial acetic acid, and 2420 mg/L Tris. The pH of the medium was adjusted to 7.0. The cells were cultured under photosynthetically active radiation (PAR) illumination of 23 mol/m²/s for 12 hours and then incubated in the dark at 22° C. for 12 hours. A 30 W halogen lamp (SZ2-CLS, Olympus, Japan) was used as the light source, and the light intensity was measured using a light power meter (8230E-82311B, ADC Corp., Japan). The number of cells per mL was determined by measuring absorbance at 750 nm (OD₇₅₀) and performing trypan blue assays for cell counting.

### Characterization

An optical microscope (NSM-3B, Samwon, South Korea) was used for optical microscopy, and a scanning electron microscope (SEM) equipped with an energy dispersive spectrometer (JSM-7610F-Plus, JEOL, Japan) was used for electron microscopy. Confocal laser scanning microscopy (CLSM) measurements were performed using a Carl Zeiss LSM 980 laser confocal microscope. The pH value was measured using a pH meter (Thermo Orion Star A211 pH Benchtop, Thermo Scientific™) equipped with a micro sensor (Mettler Toledo, Thermo Scientific™) calibrated with pH 4.01, 7.00, and 10.01 buffers (Thermo Scientific™). Organic compound analysis was performed using a nuclear magnetic resonance (NMR) spectrometer (AVANCE II 400, Bruker Biospin).

### Measurement of Chlorophyll Content and Absorption Spectrum Analysis

To extract chlorophyll, 15 mL of C. reinhardtii-CNF/Pt culture solution as a hydrogen production sample was mixed with 80% aqueous acetone (v/v) and incubated at 4° C. for 12 hours. Then, the sample was centrifuged at 3000 g for 10 minutes, and the resulting mixture was filtered using filter paper to obtain the filtrate as the crude chlorophyll extract. The absorption spectrum of the C. reinhardtii-CNF/Pt culture was measured in the range of 500-750 nm using a JASCO V-770 UV-vis spectrophotometer, and the chlorophyll content was quantified using the following equation:

$$Total chlorophyll content ( mg / g ) = \frac{CT \times V \times N}{w \times 1000}$$

$$CT = 6.63 \times A 665 + 18.08 \times A 649$$

(where V is the volume (mL) of the crude chlorophyll extract, N is the dilution factor, and W is the dry weight (g)).

### Analysis of Cell Viability

Cell viability was evaluated using CellTiterGlo® (Promega, G7572). From the hydrogen production samples, 500 μL of C. reinhardtii-CNF/Pt culture was harvested by centrifugation (900 g, 10 min). The obtained algal cells were washed three times and resuspended in 200 μL of PBS buffer, and approximately 1.83×10⁵ cells in 100 μL PBS were inoculated into a 96-well plate (Nest, 701003).

After adding 30 μL of CellTiterGlo® solution, the plate was incubated in the dark at 25° C. for 20 minutes. Absorbance at 490 nm was measured using an EnVision® 2105 (PerkinElmer), and all experiments were performed in triplicate. Cell viability was also confirmed by the trypan blue exclusion assay. A trypan blue solution (0.4% v/v) was added to the buffer at a ratio of 1:1. Live cells expelled the dye and did not appear blue, whereas dead cells retained the dye and appeared blue.

$$Cellviability ( \% ) = \frac{Dailyluminescence ( RLU )}{Initialluminescence ( RLU )} \times 100$$

### Single-Cell Analysis Using Electrochemical Scanning Microscopy (SECM)

Photosynthetic current of a single C. reinhardtii-CNF was measured using an electrochemical scanning microscope (SECM). Experiments were conducted inside a Faraday cage placed on an optical table. A nano-sized tip was positioned directly above a single cell, and the nano tip was slowly moved vertically toward the cell surface (0.1 μm/s) and the CNF surface to obtain approach curves. When the tip was sufficiently close to the cell, the applied voltage was adjusted to the value required for measuring the photosynthetic current. The tip potential was sufficiently sensitive to SECM feedback governed by diffusion. Finally, a constant height mode of SECM was applied to scan the cell over an area of 20 μm×20 μm at a scan rate of 0.4 μm/s. All electrochemical analyses were performed using a CHI920D SECM bipotentiostat (CH Instruments, USA).

Penetration of Carbon Nanofibers into C. reinhardtii

Carbon nanofibers (CNF) with an average diameter of 100 nm and a length of 7000 nm were purchased from Sigma-Aldrich (St. Louis, Missouri, USA). A CNF dispersion was prepared by adding 1 mg of CNF and 0.2 mg of CTAB (99%, Sigma-Aldrich) to 10 mL of deionized water to obtain a concentration of 100 mg/L CNF and 20 mg/L CTAB. The solution was sonicated at a fixed frequency of 40 kHz in a bath sonicator (CPX3800H-E, Branson, USA) at 25° C. for 10 hours. The solution was then centrifuged at 2000 g for 30 minutes, and the supernatant containing well-dispersed CNF was collected. To integrate C. reinhardtii with CNF, 3 mL of CNF dispersion was added to 12 mL of cell culture, and the mixed solution was passed through a borosilicate capillary tube (Sutter Instrument, USA) having an inner diameter of 0.86 mm and a length of 7 cm for 10-100 cycles.

Analysis of C. reinhardtii Penetration

Confocal laser scanning microscopy (CLSM) measurements were performed using a Carl Zeiss LSM 980 laser confocal microscope. The washed substrate was immediately stained with propidium iodide (PI; 5 g/mL) for 15 minutes and then counterstained with 4′-6-diamidino-2-phenylindole (DAPI; 5 g/mL) for 15 minutes in the dark. DAPI binds to nucleic acids and fluoresces blue when excited by a 405 nm laser, penetrating all cells. In contrast, PI enters only cells that have membrane damage considered to be penetrated and binds to nucleic acids with higher affinity than DAPI. The substrate was then imaged using CLSM.

### Formation of Metal Particles on CNF Surface

For the formation of metal particles through photosynthetic electrons, 0.5 mM silver nitrate (AgNO₃, 99%, Sigma-Aldrich), 0.25 mM copper nitrate (Cu(NO₃)₂, 99%, Sigma-Aldrich), and 0.4 mM potassium tetrachloroplatinate (K₂PtCl₄, 99%, Sigma-Aldrich) were added to the cell culture. The mixture was incubated under PAR illumination of 133 mol/m²/s at 22° C. for 1-5 hours. The light intensity was measured using a light power meter (product No. 8230E-82311B, ADC Corp., Japan). After incubation, the solution was centrifuged at 900 g for 5 minutes, and the supernatant was removed. This washing step was repeated three times using fresh TAP medium.

### Photosynthetic Hydrogen Production

A 15 mL C. reinhardtii-CNF/Pt culture was transferred to an airtight glass vial (20 mL), leaving 5 mL of headspace, and the pH was adjusted to 7.0. Then, the vial was sealed with a rubber stopper and Teflon tape to prevent gas leakage, and incubated at 22° C. under PAR illumination of 105 mol/m²/s with continuous shaking at 100 rpm for several days to induce photosynthetic hydrogen production. To detect H₂, O₂, and CO₂ contents in the headspace of the sealed vial, 100 L of gas was withdrawn at several time points using a gas-tight syringe and injected into an Agilent 7890B gas chromatograph equipped with a thermal conductivity detector (TCD) and a Carboxen 1000 12 ft column (Supelco). High-purity helium (99.999%) was used as the carrier gas for improved signal-to-noise ratio. The contents of H₂, O₂, and CO₂ were calculated based on pre-calibrated peak areas using standard gases of known concentrations. The fuel-cell-powered RC vehicle used in this study was purchased from Horizon Educational (H-racer 2.0, No. FCJJ-23). The built-in PEM fuel cell had an output power of 270 mW, an output voltage of 0.6 V (DC), and an output current of 0.45 A.

### MODE FOR CARRYING OUT THE INVENTION

### Example 1. Preparation of Cells with Inserted Carbon Nanofibers

### 1-1. Confirmation of Cell Insertion Rate by Controlling the Diameter of Carbon Nanofibers

To insert carbon nanofibers (CNF) into C. reinhardtii, the CNF dispersion was added to the cell culture, and the mixed solution was passed through a borosilicate capillary tube (Sutter Instrument, USA) having an inner diameter of 0.86 mm and a length of 7 cm for 10-100 cycles. At this time, the diameters of the carbon nanofibers were varied to 100 nm, 200 nm, and 400 nm to monitor cell insertion.

As a result, an average of 1.2±0.2 CNF penetrated the cells with a maximum efficiency of 94% when CNF with a tip diameter of 100 nm and a length of 7 m was used. Furthermore, confocal laser scanning microscopy was used to monitor penetration and recovery of the cell wall during and after the insertion process. As shown in FIG. 7, the insertion rate of carbon nanofibers with a diameter of 100 nm was the highest, and the insertion rate increased with the number of injection cycles. It was also confirmed that, after a certain period, the cells recovered and the degree of cell damage was minimal.

### 1-2. Analysis of Photosynthetic Current Extraction Function at the Single-Cell Level

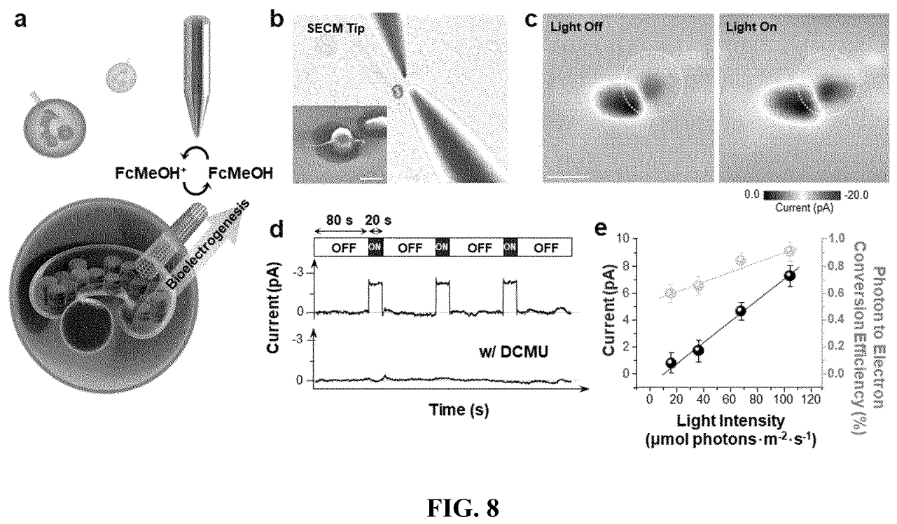

To confirm whether CNF actually secured electrical connection between the thylakoid membrane of the chloroplast and the external electrolyte solution, electrochemical scanning microscopy (SECM) was performed. A platinum nanoelectrode with a diameter of approximately 425 nm was used as the SECM tip, and extracellular photocurrent was measured at a distance of about 1 μm between the tip and C. reinhardtii-CNF. As shown in FIG. 8c, local current mapping by SECM revealed distinct current amplification under light irradiation as the extracted photoelectrons at the CNF-electrolyte interface reacted with the redox mediator. Statistical analysis on 23 cells confirmed error-free electrical connection, and extracellular photocurrent exhibited linear dependence on light intensity within the range of 18.2-108 μmol photons·m⁻²·s⁻¹, which is typical for photosynthetic research. On average, a photon-to-electron conversion efficiency of 0.7% was observed, with a maximum efficiency of 0.9% (FIG. 8e). Considering that the typical photon utilization efficiency of the Chlamydomonas genus is about 1%, almost quantitative photoelectron extraction was achieved. As shown in FIG. 8d, intermittent light chronoamperometry confirmed that current clearly started under light irradiation and abruptly ceased in the dark state, verifying that the SECM-observed current indeed originated from the algal photosystem. When the system was treated with DCMU (3-(3,4-dichlorophenyl)-1,1-dimethylurea), known to block the photosynthetic electron transport chain, the extracellular current completely stopped, confirming that the measured current was extracted from the algal photosystem. Thus, C. reinhardtii-CNF according to the present invention functions as an eco-friendly cellular photovoltaic power plant providing 9.5 pW per cell.

### Example 2. Formation of Metal Catalyst Particles Using Cells with Inserted Carbon Nanofibers

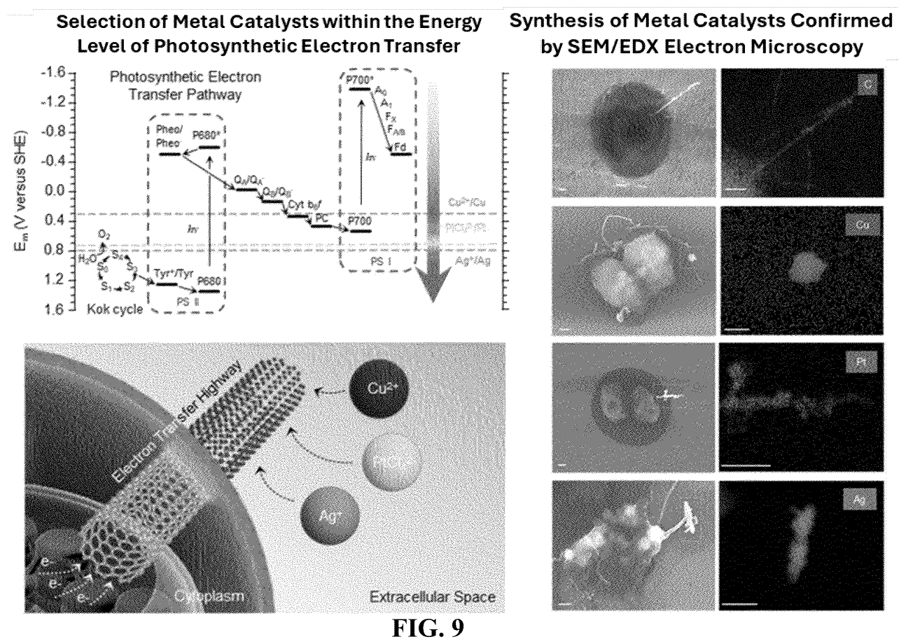

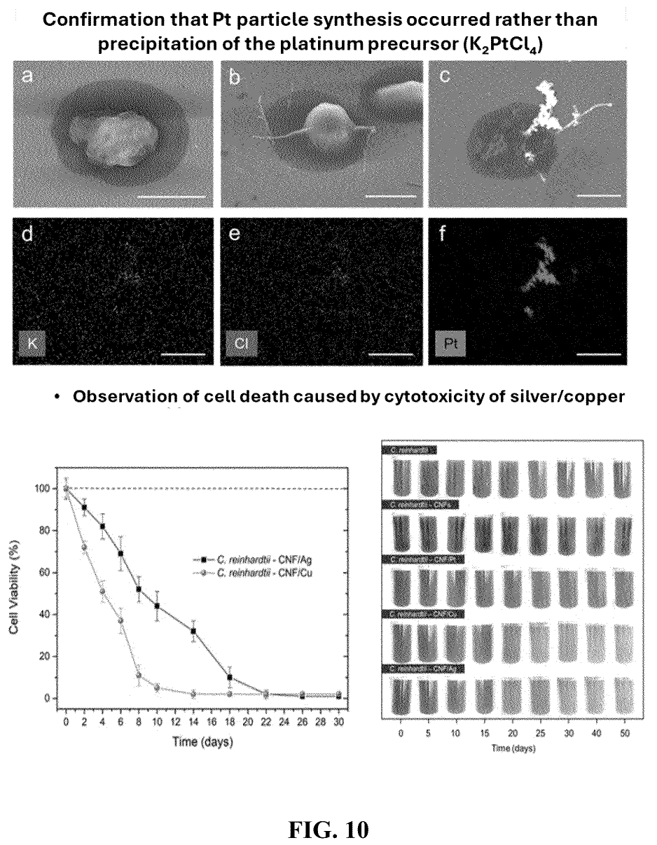

The voltage environment of the electron carrier compounds in the thylakoid Z-scheme is shown in FIG. 9. In the C. reinhardtii-CNF power plant according to the present invention, the excited compounds in photosystem I responsible for photocurrent emission have a more negative standard redox potential than Cu, Ag, and Pt cations. The difference in redox potential enabled the synthesis of metal nanoparticles on the external terminal of CNF using photoelectrons as reducing agents. As shown in FIG. 9, Cu, Ag, and Pt were clearly and selectively formed on the surface of CNF. However, synthesized Cu and Ag particles were expected to be unsuitable for long-term use with algal-CNF due to cytotoxicity (FIG. 10).

### Example 3. Hydrogen Production Based on Metal Catalyst for Hydrogen Evolution

### 3-1. Oxygen Removal Through Oxygen Reduction Reaction and Hydrogen Generation

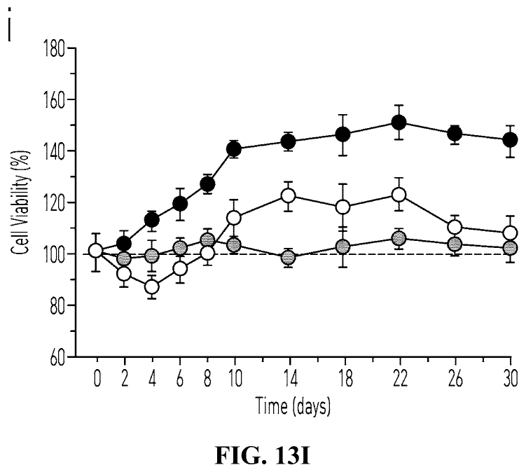

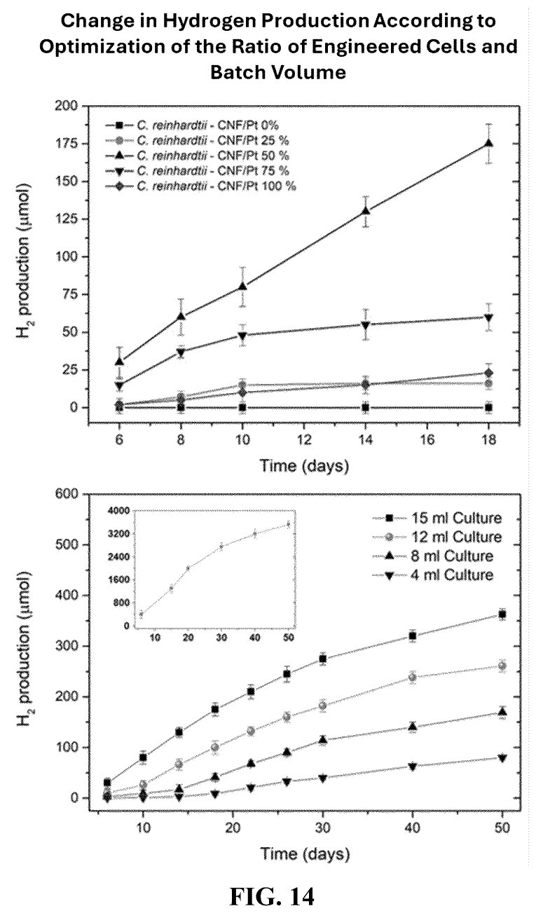

Photosynthetic hydrogen production was confirmed using the fully designed C. reinhardtii-CNF/Pt hybrid power plant. A sealed batch reactor containing C. reinhardtii-CNF/Pt was prepared, and the gas fraction in the headspace where hydrogen accumulated was monitored. Various ratios of unmanipulated wild-type algal cells and manipulated algal cells were tested to examine the change in hydrogen yield, and it was confirmed that the optimal hydrogen production was achieved at a 1:1 ratio (FIG. 14).

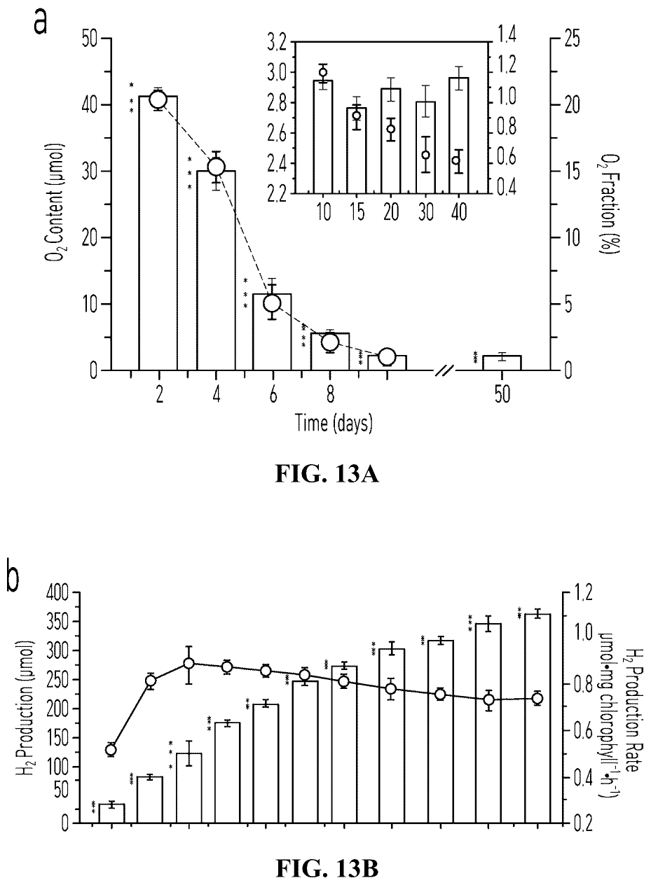

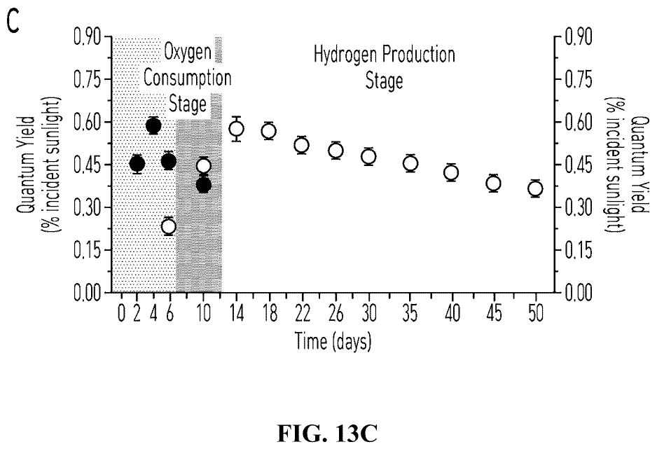

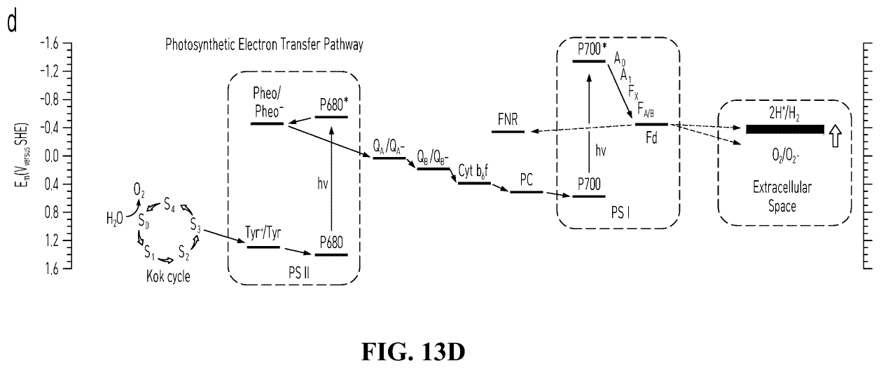

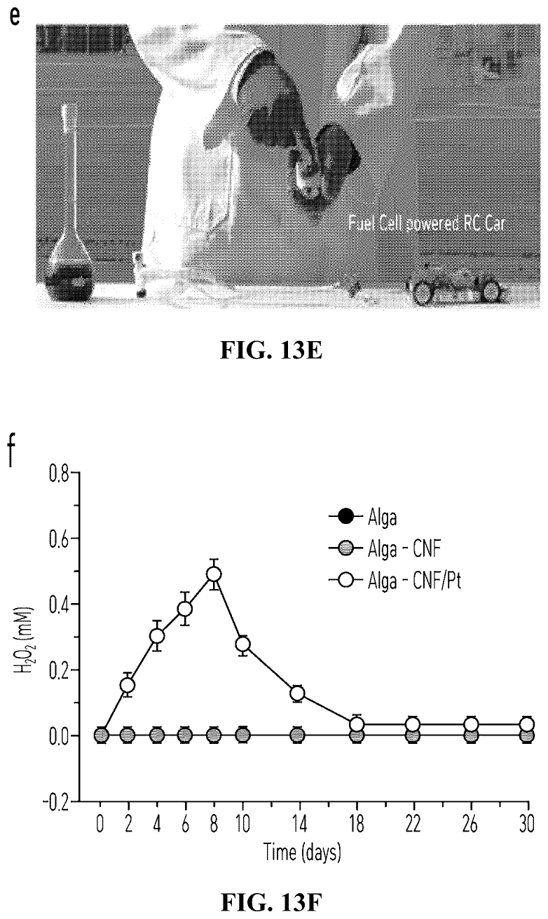

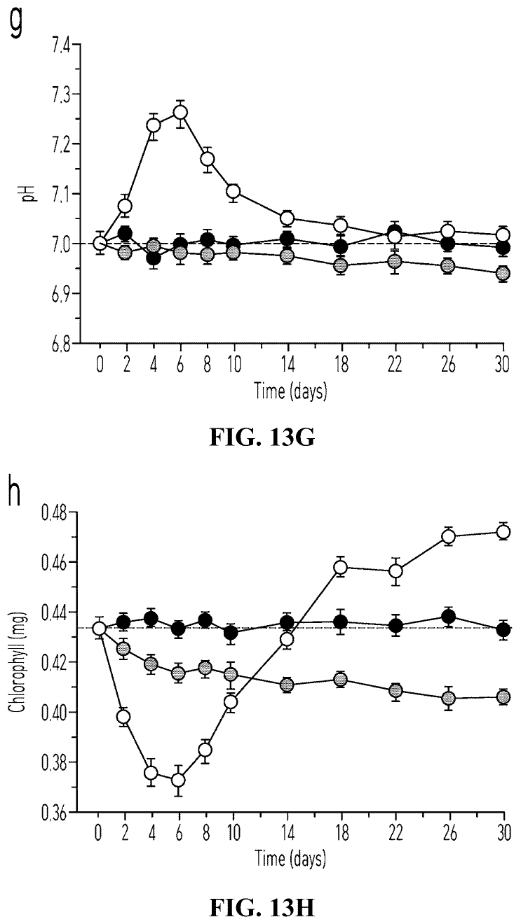

Extracted photoelectrons were transferred as reducing equivalents to the Pt surface, where two chemical species that can be reduced at the potential provided by the photosystem—protons and dioxygen molecules—were present in the solution. These two competing reactions, hydrogen evolution and oxygen reduction, have formal potentials of −0.41 and −0.33 V versus SHE, respectively (FIG. 13d). Since platinum is an excellent catalyst for both reactions and has a lower cathodic potential for oxygen reduction, the oxygen reduction reaction occurs prior to hydrogen evolution. As shown in FIGS. 13a-13c, during the first four days of operation, the extracellular current was consumed only for oxygen reduction. Because the formal potential for oxygen reduction is a Nernstian function of the O₂/O₂⁻ concentration ratio, the decrease in oxygen concentration shifted the formal potential cathodically. After four days, when the oxygen concentration dropped below 5%, hydrogen was first detected in the headspace on the sixth day, and the reduction pathway thereafter proceeded toward hydrogen production (FIG. 13c). In the competition between oxygen reduction and hydrogen evolution, the system of the present invention automatically regulated the oxygen concentration in the headspace to below 1.0% without external assistance. During the initial stage of artificial photosynthesis, a surge of oxygen reduction generated detectable amounts of hydrogen peroxide (FIG. 13f), which decomposed naturally over time during the hydrogen production stage. A transient increase in pH occurred simultaneously with peroxide formation due to proton consumption in the oxygen reduction reaction (FIG. 13g). Decreases in chlorophyll concentration (FIG. 13h) and cell viability (FIG. 13i) corresponded to peroxide generation and pH increase. However, as peroxide decreased and pH returned to physiological levels, both factors fully recovered to sustainable levels during the oxygen-depleted hydrogen production phase.

With minimal electron diversion to competing oxygen reduction, hydrogen production accelerated from the sixth day onward. The highest production rate of 0.8 mol H₂·mg chlorophyll⁻¹·h⁻¹ was recorded on the tenth day and maintained up to the fiftieth day, producing 363 mol of hydrogen from a 15 mL batch (FIG. 13b). A high quantum yield of 0.45±0.09% for the overall reaction was recorded throughout the entire experiment (FIG. 13c). The observed photon-to-hydrogen quantum yield exceeded the reported carbon fixation efficiency of algae and plants (typically 0.2-1.6%), indicating that the photoelectron relay chain of the algal cell power plant according to the present invention was highly efficient.

The continuous photosynthetic hydrogen generation exceeding 50 days observed in the present invention is one of the longest-lasting systems, and its hydrogen generation rate is among the highest reported. Moreover, the fuel production rate increased linearly with the batch volume (FIG. 14). A 150 mL batch produced up to 4 mmol of H₂, which is an unusually large amount for a laboratory-scale experiment. With a large amount of accumulated fuel and a characteristic of self-regulated minimum oxygen concentration, electrical power was supplied directly to a fuel-cell-powered RC vehicle by gas injection from the headspace of the batch reactor, demonstrating smooth driving comparable to that of a vehicle powered by pure hydrogen (FIG. 13e). In particular, it was confirmed that the absence of additional purification or fuel separation steps prior to use as fuel provides a significant advantage.

### 3-2. Continuous Electron Supply System Through Photosynthetic CO₂ Fixation of Cells

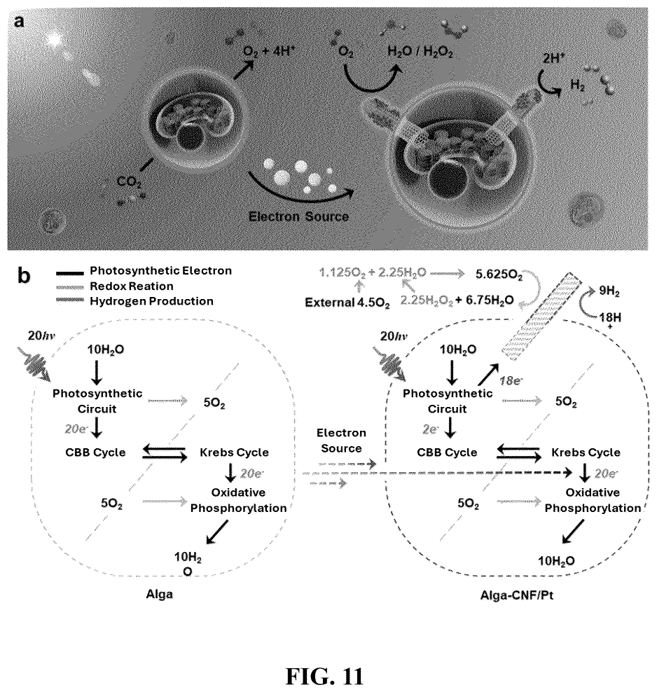

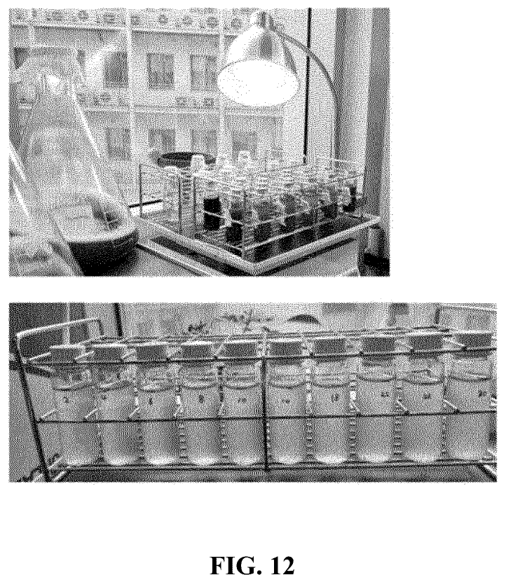

As shown in FIG. 11, in the engineered algal cells, photoelectrons were extracted outside the cell through the wire, so only a small portion entered the Calvin-Benson-Bassham (CBB) cycle, and therefore, an additional electron source was required for respiration. Although acetate molecules in the growth medium were used as the electron source, it was confirmed that acetate was depleted after four days of cultivation. However, even after the depletion of acetate, the amount of hydrogen production did not decrease, and a decrease in the CO₂ concentration in the headspace was observed. It was therefore presumed that carbon fixation products (for example, polysaccharides) secreted by unmanipulated algal cells served as electron donors.

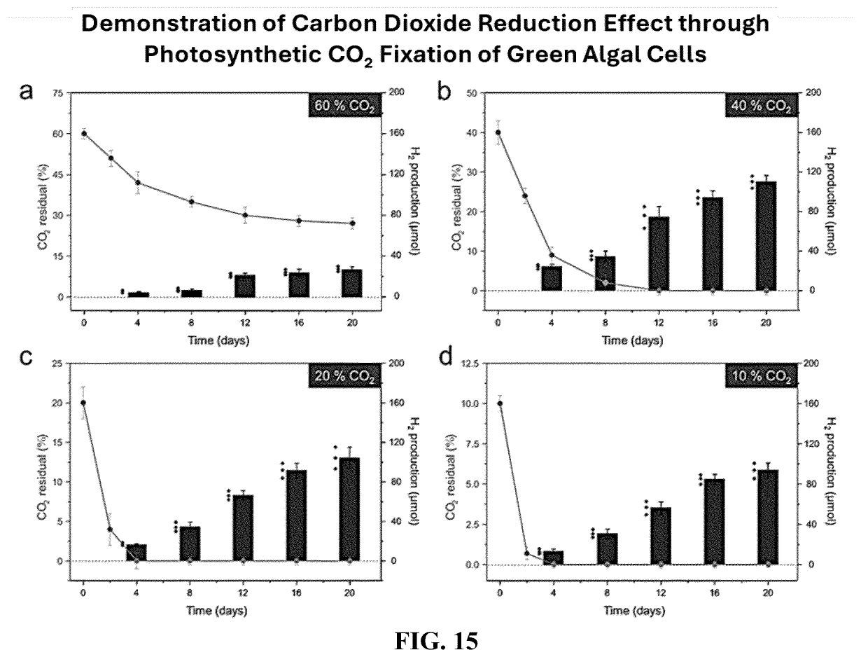

Accordingly, the reaction conditions were further modified for hydrogen generation without consuming acetate as the electron donor. C. reinhardtii-CNF/Pt was cultured in an acetate-free medium, and the headspace was charged with CO₂ at various concentrations. As designed, the clean algal cells fixed CO₂ into storage compounds that could be shared with C. reinhardtii-CNF/Pt, demonstrating that long-term hydrogen production was possible using only CO₂ and sunlight (FIG. 15). Although acetate also functions as a growth promoter in Chlamydomonas cultures in addition to serving as an electron donor, the hydrogen production rate in the acetate-free medium was about 60% of that with acetate. Nevertheless, converting solar energy into fuel while consuming only CO₂ and water as electron sources represents a sufficiently excellent result.

### INDUSTRIAL APPLICABILITY

According to the present invention, due to the dual functionality of platinum that catalyzes both hydrogen evolution and oxygen reduction reactions, the oxygen concentration in the headspace of the batch reactor containing algal cells-CNF/Pt was automatically regulated to a minimum level. Accordingly, hydrogen fuel could be utilized without separation or purification of oxygen, and the operation of a fuel-cell-powered RC vehicle by direct injection from the headspace of the batch reactor demonstrated the effect of the present invention. Furthermore, hydrogen production increased linearly with the batch volume. With proven scalability, as well as cost and spatial efficiency of energy production, the present invention can make a significant contribution to future renewable energy research fields.

1-20. (canceled)

21. A method for producing hydrogen, comprising irradiating light to a mixture of a cell comprising a chloroplast and manipulated such that a wire is inserted from outside the cell to the inside of the chloroplast, and a metal ion solution, thereby producing hydrogen.

22. The method for producing hydrogen according to claim 21, wherein the method comprises:
a) extracting photosynthetic electrons generated inside the cell by light through the wire to the outside of the cell;
b) forming metal ions on the surface of the wire by the extracted photosynthetic electrons; and
c) generating hydrogen on the surface of the wire where the metal ions are formed.

23. The method for producing hydrogen according to claim 21, wherein the metal ion is selected from the group consisting of gold, platinum, copper, silver, iron, nickel, cobalt, and lead.

24. The method for producing hydrogen according to claim 21, wherein the light is irradiated at an intensity of 0.1 to 15 mW/cm².

25. The method for producing hydrogen according to claim 21, wherein the light is irradiated for 5 to 20 hours.

26. The method for producing hydrogen according to claim 21, wherein oxygen in the metal ion solution is removed by an oxygen reduction reaction in the initial stage of step c).

27. The method for producing hydrogen according to claim 21, wherein after oxygen in the metal ion solution is removed in step c), a hydrogen generation reaction occurs.

28. The method for producing hydrogen according to claim 21, wherein the wire is made of a carbon nanomaterial or a conductive metal material.

29. The method for producing hydrogen according to claim 28, wherein the conductive metal is selected from the group consisting of gold, platinum, copper, silver, iron, nickel, cobalt, and lead.

30. The method for producing hydrogen according to claim 21, wherein the cell is selected from the group consisting of algal cells, plant cells, and artificial cells containing a chloroplast.

31-33. (canceled)

34. The method for producing hydrogen according to claim 21, wherein the wire contacts or penetrates the thylakoid membrane of the chloroplast.

35. The method for producing hydrogen according to claim 21, wherein the diameter of the wire is 100 to 400 nm.

36. The method for producing hydrogen according to claim 21, wherein the length of the wire is 5 to 30 m.

37. The method for producing hydrogen according to claim 21, wherein photosynthetic electrons generated inside the cell by light are transferred to the outside of the cell through the wire.

38. The method for producing hydrogen according to claim 21, wherein the cell is produced by the following steps:
a) preparing a dispersion comprising a cell culture including chloroplasts and a wire;
b) adding the dispersion containing the wire to the cell culture to prepare a mixed solution; and
c) inserting the wire into the inside of the cell through physical collision between the cells and the wire in the mixed solution.

39. The method for producing hydrogen according to claim 38, wherein step (c) is performed by fluid injection or ball milling.
